(19) United States
(12) Patent Application Publication
(10) Pub. No.: US 2025/0226107 A1
(43) Pub. Date: Jul. 10, 2025

(54) MACHINE LEARNING MODELS FOR ADDISON'S DISEASE RISK ANALYSIS

(71) Applicant: IDEXX Laboratories, Inc., Westbrook, ME (US)

(72) Inventors: Yu ZHANG, Pittsburgh, PA (US); Mary Alexis SEGUIN, North Freedom, WI (US); Margaret CANNING, Richmond, TX (US); David KINCAID, Eu Claire, WI (US)

(21) Appl. No.: 19/009,409

(22) Filed: Jan. 3, 2025

Related U.S. Application Data
(60) Provisional application No. 63/617,530, filed on Jan. 4, 2024.

(51) Int. Cl. G16H 50/30; A61B 5/00; G16H 10/60; G16H 20/17; G16H 50/20

(52) U.S. Cl. CPC G16H 50/30; G16H 10/60; G16H 20/17; G16H 50/20; A61B 5/7267; A61B 5/7275

(57) ABSTRACT

Systems and methods for assessing the risk of Addison's disease are described. An ensemble of diagnostic models is trained on a first set of medical training data. A knowledge based diagnostic model is trained on a second set of medical training data. When new patient data is received, the ensemble of diagnostic models is used to determine whether a risk for Addison's disease is indicated for the new patient data. If the ensemble of diagnostic models indicates a risk for Addison's disease for the new patient data, the risk of Addison's disease is assessed using the knowledge based diagnostic model. The knowledge based diagnostic model interacts with a user interface is used to acquire additional information from a clinician. The user interface provides an assessment of the risk of Addison's disease for the new patient data, and guidance for further testing and treatment.

### CROSS-REFERENCE TO RELATED APPLICATIONS

This application claims the benefit of U.S. Application No. 63/617,530, filed Jan. 4, 2024, the entire disclosure of which is hereby incorporated herein by reference.

### TECHNICAL FIELD

The present disclosure generally relates to diagnostic models for human and veterinary applications, and more specifically relates to machine learning models for analyzing and predicting the risk of Addison's disease.

### BACKGROUND

Hypoadrenocorticism (Addison's disease) is a rare but serious condition in dogs who have deficiencies in adrenal hormones (i.e. cortisol, aldosterone). In primary Addison's disease, adrenal hormone deficiency is primarily due to destruction (immune-mediated) of the adrenal cortices. Decreased cortisol resulting from destruction of the zona fasciculata and reticularis is the most consistent finding in Addison's disease. This glucocorticoid deficiency inhibits a patient's ability to respond to internal and external stress appropriately. Clinical signs of cortisol deficiency include lethargy, inappetence, and, in some cases, chronic or intermittent vomiting and/or diarrhea. These signs may manifest more strongly following stressful events.

In the majority of cases with primary Addison's disease, the zona glomerulosa is also affected, resulting in mineralocorticoid deficiency in addition to glucocorticoid deficiency. Mineralocorticoid deficiency leads to electrolyte disturbances including hyperkalemia and low sodium. Patients with significant electrolyte abnormalities may present acutely with hypovolemia and shock. The “typical” form of primary Addison's disease includes both glucocorticoid and mineralocorticoid deficiencies. Patients with an “atypical” form of Addison's disease may present with normal electrolytes, suggesting a deficiency of glucocorticoids with normal mineralocorticoid levels. In the absence of classical electrolyte changes, laboratory findings may be subtle (e.g., lack of a stress leukogram) or similar to those seen in other more common conditions (e.g., decreases in albumin and cholesterol suggestive of GI or liver disease).

For this reason, Addison's disease is sometimes referred to as the “great pretender” due to the variability of clinical signs, laboratory results, and patient history. Patients with Addison's disease may present with non-specific clinical signs that wax and wane over months to years, or they may present in an acute hypovolemic shock crisis. Laboratory abnormalities may mimic changes seen in other diseases such as liver disease, gastrointestinal disease, and acute kidney injury, or may be very subtle and nonspecific. In general practice, Addison's disease is often missed as a potential differential diagnosis for patients with gastrointestinal or non-specific clinical signs. If a patient is exhibiting non-specific signs over a long period of time, a veterinarian may waste a great deal of time and resources trying to treat the variety of clinical presentations that a patient may be exhibiting without ever recognizing or addressing the underlying Addison's disease.

Veterinarians generally rely on changes seen in minimum database laboratory tests to recognize the possibility of Addison's disease. Additional diagnostic/laboratory testing is required to actually diagnose Addison's disease. Veterinarians are likely to rely on electrolyte changes as a primary trigger to consider Addison's disease as a differential diagnosis. However, these electrolyte changes are inconsistently seen in patients with Addison's disease and may only be present with more advanced disease or when the patient is in crisis. Veterinarians may miss the subtler patterns seen within a complete blood count (CBC) and/or biochemistry laboratory tests in early-stage Addison's disease. In addition, veterinarians are less likely to include screening for resting cortisol tests, which could aid in earlier recognition, as part of their diagnostic work-up of patients with gastrointestinal or non-specific clinical signs. Often, subtle clinical signs go unrecognized until a stressful event (e.g., boarding, concurrent illness, routine elective procedures such as a dentistry) triggers an acute crisis. These crises can be severe, requiring hospitalization. In acute cases, or if a patient remains undiagnosed, Addison's disease is life threatening. The ability to detect Addison's disease prior to the development of an Addisonian crisis is essential to instituting lifesaving treatment before the patient becomes critically ill.

Since the initial symptoms of Addison's are non-specific and can fit other conditions, when taken separately, each symptom may be discounted or explained away, whilst the patient remains undiagnosed. As it is easy for a veterinarian to miss the signs of Addison's disease, especially in the early stages, there exists a need for a new approach that utilizes machine learning to assess the risk for Addison's disease using basic laboratory tests and clinical observations.

### SUMMARY

At least the above-discussed needs are addressed, and technical solutions are achieved in the art, by various aspects of the present disclosure.

Some aspects of the present disclosure pertain to a method executed by a programmed data processing device system comprising receiving first medical training data; training a plurality of first machine learning models on the received first medical training data to generate an ensemble of diagnostic models; receiving second medical training data; training a second machine learning model on the received second medical training data to generate a knowledge based diagnostic model; receiving new patient data; determining whether the ensemble of diagnostic models indicates a risk for Addison's disease for the new patient data; and, in a case where the ensemble of diagnostic models indicates a risk for Addison's disease for the new patient data, assessing the risk of Addison's disease using the knowledge based diagnostic model.

In some aspects of the present disclosure, the ensemble of diagnostic models includes one or more of a decision tree model, a random forest model, a neural network, a support vector machine, or a nearest neighbor classifier.

In some aspects of the present disclosure, training the plurality of first machine learning models includes selecting at least a first machine learning model and a second machine learning model for the ensemble of diagnostic models, wherein the second machine learning model is different from the first machine learning model; training the first machine learning model using a first subset of the received first medical training data to generate a first model of the ensemble of diagnostic models; and training the second machine learning model using a second subset of the received first medical training data to generate a second model of the ensemble of diagnostic models, wherein the second subset of the received first medical training data is different from the first subset of the received first medical training data. The risk for Addison's disease for the new patient data is determined based on outputs of the first machine learning model and the second machine learning model. In some aspects of the present disclosure, it is determined that the ensemble of diagnostic models indicates a risk for Addison's disease for the new patient data in a case where the outputs of either the first machine learning model or the second machine learning indicate the risk for Addison's disease for the new patient data.

In some aspects of the present disclosure, training the second machine learning model includes generating an expert model including a plurality of rules based on domain knowledge included in the received second medical training data; refining the plurality of rules in the expert model based on patient data included in the received second medical training data; and generating the knowledge based diagnostic model based on the refined plurality of rules.

In some aspects of the present disclosure, the knowledge based diagnostic model includes one or more of a rules based model and a data driven machine learning model.

In some aspects of the present disclosure, assessing the risk of Addison's disease using the knowledge based diagnostic model further includes displaying, on a user interface, a series of prompts to receive further patient medical data; and assessing the risk of Addison's disease based the received further patient medical data in response to the series of prompts. In some aspects of the present disclosure, each subsequent prompt of the series of prompts is dynamically selected using the knowledge based diagnostic model based on a response to a preceding prompt of the series of prompts.

In some aspects of the present disclosure, the knowledge based diagnostic model is an interactive model, and assessing the risk of Addison's disease includes interacting with a user using the knowledge based diagnostic model to provide guidance on diagnosis and treatment of Addison's disease.

In some aspects of the present disclosure, the method further comprises, in response to determining that the assessed risk of Addison's disease is above a predefined threshold, administering at least one of intravenous fluids, glucocorticoids, or mineralocorticoids.

In some aspects of the present disclosure, a diagnostic system for assessing the risk of Addison's disease comprises a memory configured to store instructions, and a processor communicatively connected to the memory and configured to execute the stored instructions. The processor executes the stored instructions to receive first medical training data; train a plurality of first machine learning models on the received first medical training data to generate an ensemble of diagnostic models; receive second medical training data; train a second machine learning model on the received second medical training data to generate a knowledge based diagnostic model; receive new patient data; determine whether the ensemble of diagnostic models indicates a risk for Addison's disease for the new patient data; and, in a case where the ensemble of diagnostic models indicates a risk for Addison's disease for the new patient data, assess the risk of Addison's disease using the knowledge based diagnostic model.

In some aspects of the present disclosure, a non-transitory computer readable storage medium is configured to store a program that executes the diagnostic methods discussed above.

Subsets or combinations of various aspects of the disclosure described above provide further aspects of the disclosure.

### BRIEF DESCRIPTION OF THE DRAWINGS

It is to be understood that the attached drawings are for purposes of illustrating aspects of various aspects of the disclosure and may include elements that are not to scale. It is noted that like reference characters in different figures refer to the same objects.

FIG. 1 shows a computing device system, according to some aspects of the disclosure.

FIG. 2 shows another computing device system, according to some aspects of the disclosure.

FIG. 3 shows a block diagram of a diagnostic system for assessing the risk of Addison's disease, according to some aspects of the disclosure.

FIG. 4 shows a flowchart of a machine learning based Addison's disease risk assessment method, according to some aspects of the disclosure.

FIG. 5 shows a flowchart of a method of generating machine learning models for assessing the risk of Addison's disease, according to some aspects of the disclosure.

FIG. 6 shows a flowchart of an interactive machine learning based Addison's disease risk assessment method, according to some aspects of the disclosure.

### DETAILED DESCRIPTION

In some aspects of the disclosure, the computer systems described herein execute methods for implementing machine learning models that predict the risk of Addison's disease. It should be noted that the aspects or embodiments of the present disclosure are not limited to these or any other examples provided herein, which are referred to for purposes of illustration only. In this regard, in the descriptions herein, certain specific details are set forth in order to provide a thorough understanding of various aspects of the disclosure. However, one skilled in the art will understand that the invention may be practiced at a more general level without one or more of these details. In other instances, well-known structures have not been shown or described in detail to avoid unnecessarily obscuring descriptions of various aspects of the disclosure.

Any reference throughout this specification to one “aspect” or “embodiment”, an “aspect” or “embodiment,” an example “aspect” or “embodiment,”, an illustrated “aspect” or “embodiment,” a particular “aspect” or “embodiment,” and the like means that a particular feature, structure, or characteristic described in connection with the aspect or embodiment is included in at least one aspect or embodiment. Thus, any appearance of the phrase in one “aspect” or “embodiment,” in an “aspect” or “embodiment,” in an example “aspect” or “embodiment,” in this illustrated “aspect” or “embodiment,” in this particular “aspect” or “embodiment,” or the like in this specification is not necessarily all referring to one aspect or embodiment or a same aspect or embodiment. Furthermore, the particular features, structures or characteristics of different aspects or embodiments of the disclosure may be combined in any suitable manner to form one or more other aspects or embodiments of the disclosure. Further, the term aspect or embodiment may be used interchangeably.

Unless otherwise explicitly noted or required by context, the word “or” is used in this disclosure in a non-exclusive sense. In addition, unless otherwise explicitly noted or required by context, the word “set” is intended to mean one or more. For example, the phrase, “a set of objects” means one or more of the objects.

In the following description, some aspects of the disclosure may be implemented at least in part by a data processing device system configured by a software program. Such a program may equivalently be implemented as multiple programs, and some or all of such software program(s) may be equivalently constructed in hardware.

Further, the phrase “at least” is or may be used herein at times merely to emphasize the possibility that other elements may exist beside those explicitly listed. However, unless otherwise explicitly noted (such as by the use of the term “only”) or required by context, non-usage herein of the phrase “at least” nonetheless includes the possibility that other elements may exist besides those explicitly listed. For example, the phrase, ‘based at least on A’ includes A as well as the possibility of one or more other additional elements besides A. In the same manner, the phrase, ‘based on A’ includes A, as well as the possibility of one or more other additional elements besides A. However, the phrase, ‘based only on A’ includes only A. Similarly, the phrase ‘configured at least to A’ includes a configuration to perform A, as well as the possibility of one or more other additional actions besides A. In the same manner, the phrase ‘configured to A’ includes a configuration to perform A, as well as the possibility of one or more other additional actions besides A. However, the phrase, ‘configured only to A’ means a configuration to perform only A.

The word “device,” the word “machine,” the word “system,” and the phrase “device system” all are intended to include one or more physical devices or sub-devices (e.g., pieces of equipment) that interact to perform one or more functions, regardless of whether such devices or sub-devices are located within a same housing or different housings. However, it may be explicitly specified according to various aspects of the disclosure that a device or machine or device system resides entirely within a same housing to exclude aspects of the disclosure where the respective device, machine, system, or device system resides across different housings. The word “device” may equivalently be referred to as a “device system” in some aspects of the disclosure.

The phrase “derivative thereof” and the like is or may be used herein at times in the context of a derivative of data or information merely to emphasize the possibility that such data or information may be modified or subject to one or more operations. For example, if a device generates first data for display, the process of converting the generated first data into a format capable of being displayed may alter the first data. This altered form of the first data may be considered a derivative of the first data. For instance, the first data may be a one-dimensional array of numbers, but the display of the first data may be a color-coded bar chart representing the numbers in the array. For another example, if the above-mentioned first data is transmitted over a network, the process of converting the first data into a format acceptable for network transmission or understanding by a receiving device may alter the first data. As before, this altered form of the first data may be considered a derivative of the first data. For yet another example, generated first data may undergo a mathematical operation, a scaling, or a combining with other data to generate other data that may be considered derived from the first data. In this regard, it can be seen that data is commonly changing in form or being combined with other data throughout its movement through one or more data processing device systems, and any reference to information or data herein is intended to include these and like changes, regardless of whether or not the phrase “derivative thereof” or the like is used in reference to the information or data, unless otherwise required by context. As indicated above, usage of the phrase “or a derivative thereof” or the like merely emphasizes the possibility of such changes. Accordingly, the addition of or deletion of the phrase “or a derivative thereof” or the like should have no impact on the interpretation of the respective data or information. For example, the above-discussed color-coded bar chart may be considered a derivative of the respective first data or may be considered the respective first data itself.

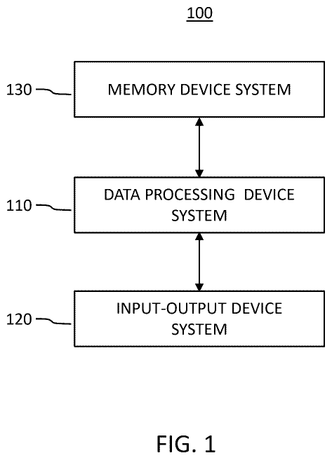

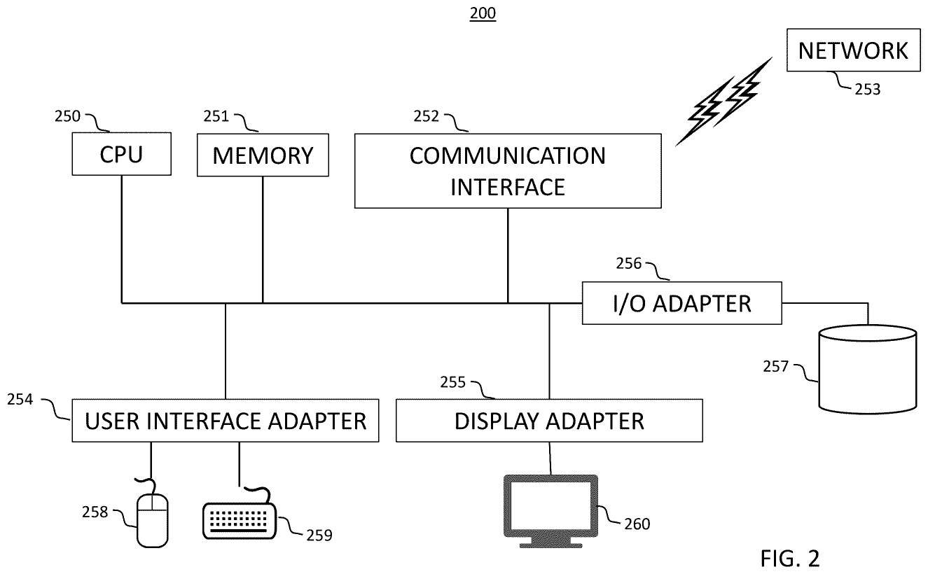

The term “program” in this disclosure should be interpreted to include one or more programs including a set of instructions or modules that may be executed by one or more components in a system, such as a controller system or data processing device system, to cause the system to perform one or more operations. The set of instructions or modules may be stored by any kind of memory device, such as those described subsequently with respect to the memory device system 130, 251, or both, shown in FIGS. 1 and 2, respectively. In addition, this disclosure may describe or similarly describe that the instructions or modules of a program are configured to cause the performance of an action. The phrase “configured to” in this context is intended to include at least (a) instructions or modules that are presently in a form executable by one or more data processing devices to cause performance of the action (e.g., in the case where the instructions or modules are in a compiled and unencrypted form ready for execution), and (b) instructions or modules that are presently in a form not executable by the one or more data processing devices, but could be translated into the form executable by the one or more data processing devices to cause performance of the action (e.g., in the case where the instructions or modules are encrypted in a non-executable manner, but through performance of a decryption process, would be translated into a form ready for execution). Such descriptions should be deemed to be equivalent to describing that the instructions or modules are configured to cause the performance of the action. The word “module” may be defined as a set of instructions. The word “program” and the word “module” may each be interpreted to include multiple sub-programs or multiple sub-modules, respectively. In this regard, reference to a program or a module may be considered to refer to multiple programs or multiple modules.

Further, it is understood that information or data may be operated upon, manipulated, or converted into different forms as it moves through various devices or workflows. In this regard, unless otherwise explicitly noted or required by context, it is intended that any reference herein to information or data includes modifications to that information or data. For example, “data X” may be encrypted for transmission, and a reference to “data X” is intended to include both its encrypted and unencrypted forms, unless otherwise required or indicated by context. However, non-usage of the phrase “or a derivative thereof” or the like nonetheless includes derivatives or modifications of information or data just as usage of such a phrase does, as such a phrase, when used, is merely used for emphasis.

Further, the phrase “graphical representation” used herein is intended to include a visual representation presented via a display device system and may include computer-generated text, graphics, animations, or one or more combinations thereof, which may include one or more visual representations originally generated, at least in part, by an image-capture device.

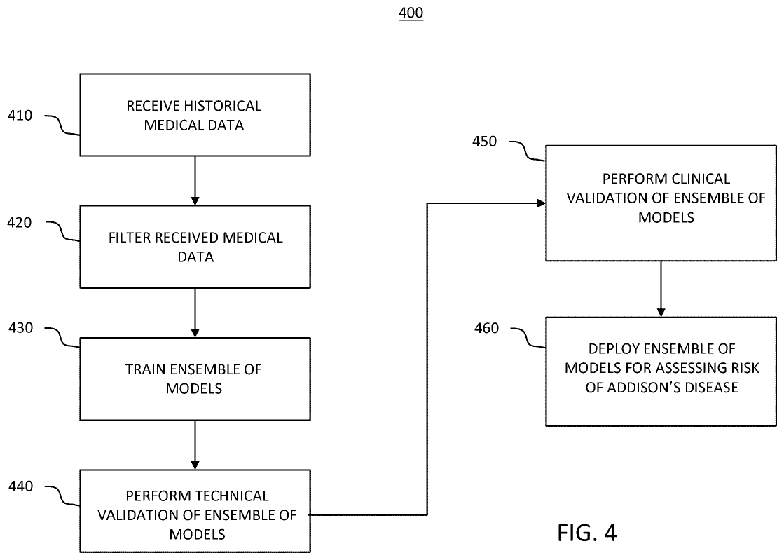

Further still, example methods are described herein with respect to FIGS. 4-6. Such figures are described to include blocks associated with computer-executable instructions. It should be noted that the respective instructions associated with any such blocks herein need not be separate instructions and may be combined with other instructions to form a combined instruction set. The same set of instructions may be associated with more than one block. In this regard, the block arrangements shown in method FIGS. 4-6 herein are not limited to an actual structure of any program or set of instructions or required ordering of method tasks, and such method FIGS. 4-6, according to some aspects of the disclosure, merely illustrate the tasks that instructions are configured to perform, for example upon execution by a data processing device system in conjunction with interactions with one or more other devices or device systems.

FIG. 1 schematically illustrates a system 100 according to some aspects of the disclosure. In some aspects of the disclosure, the system 100 may be a computing device 200 (as shown in FIG. 2). In some aspects of the disclosure, the system 100 includes a data processing device system 110, an input-output device system 120, and a processor-accessible memory device system 130. The processor-accessible memory device system 130 and the input-output device system 120 are communicatively connected to the data processing device system 110.

The data processing device system 110 includes one or more data processing devices that implement or execute, in conjunction with other devices, such as one or more of those in the system 100, control programs associated with some of the various aspects of the disclosure. Each of the phrases “data processing device,” “data processor,” “processor,” and “computer” is intended to include any data processing device, such as a central processing unit (“CPU”), a circuit, a field programmable gate array (FPGA), a desktop computer, a laptop computer, a mainframe computer, a tablet computer, a personal digital assistant, a cellular phone, and any other device configured to process data, manage data, or handle data, whether implemented with electrical, magnetic, optical, biological components, or the like.

The memory device system 130 includes one or more processor-accessible memory devices configured to store information, including the information needed to execute the control programs associated with some of the various aspects of the disclosure. The memory device system 130 may be a distributed processor-accessible memory device system including multiple processor-accessible memory devices communicatively connected to the data processing device system 110 via a plurality of computers and/or devices. On the other hand, the memory device system 130 need not be a distributed processor-accessible memory system and, consequently, may include one or more processor-accessible memory devices located within a single data processing device.

Each of the phrases “processor-accessible memory” and “processor-accessible memory device” is intended to include any processor-accessible data storage device, whether volatile or nonvolatile, electronic, magnetic, optical, or otherwise, including but not limited to, registers, floppy disks, hard disks, Compact Discs, DVDs, flash memories, ROMs (Read-Only Memory), and RAMs (Random Access Memory). In some aspects of the disclosure, each of the phrases “processor-accessible memory” and “processor-accessible memory device” is intended to include a non-transitory computer-readable storage medium. In some aspects of the disclosure, the memory device system 130 can be considered a non-transitory computer-readable storage medium system.

The phrase “communicatively connected” is intended to include any type of connection, whether wired or wireless, between devices, data processors, or programs in which data may be communicated. Further, the phrase “communicatively connected” is intended to include a connection between devices or programs within a single data processor, a connection between devices or programs located in different data processors, and a connection between devices not located in data processors at all. In this regard, although the memory device system 130 is shown separately from the data processing device system 110 and the input-output device system 120, one skilled in the art will appreciate that the memory device system 130 may be located completely or partially within the data processing device system 110 or the input-output device system 120. Further in this regard, although the input-output device system 120 is shown separately from the data processing device system 110 and the memory device system 130, one skilled in the art will appreciate that such system may be located completely or partially within the data processing system 110 or the memory device system 130, depending upon the contents of the input-output device system 120. Further still, the data processing device system 110, the input-output device system 120, and the memory device system 130 may be located entirely within the same device or housing or may be separately located, but communicatively connected, among different devices or housings. In the case where the data processing device system 110, the input-output device system 120, and the memory device system 130 are located within the same device, the system 100 of FIG. 1 can be implemented by a single application-specific integrated circuit (ASIC) in some aspects of the disclosure.

The input-output device system 120 may include a mouse, a keyboard, a touch screen, another computer, or any device or combination of devices from which a desired selection, desired information, instructions, or any other data is input to the data processing device system 110. The input-output device system 120 may include any suitable interface for receiving information, instructions or any data from other devices and systems described in various ones of the aspects of the disclosure.

The input-output device system 120 also may include an image generating device system, a display device system, a speaker device system, a processor-accessible memory device system, or any device or combination of devices to which information, instructions, or any other data is output from the data processing device system 110. In this regard, if the input-output device system 120 includes a processor-accessible memory device, such memory device may or may not form part or all of the memory device system 130. The input-output device system 120 may include any suitable interface for outputting information, instructions or data to other devices and systems described in various ones of the aspects of the disclosure. In this regard, the input-output device system may include various other devices or systems described in various aspects of the disclosure.

FIG. 2 shows an example of a computing device system 200, according to some aspects of the disclosure. The computing device system 200 may include a processor 250, corresponding to the data processing device system 110 of FIG. 1, in some aspects of the disclosure. The memory 251, input/output (I/O) adapter 256, and non-transitory storage medium 257 may correspond to the memory device system 130 of FIG. 1, according to some aspects of the disclosure. The user interface adapter 254, mouse 258, keyboard 259, display adapter 255, and display 260 may correspond to the input-output device system 120 of FIG. 1, according to some aspects of the disclosure. The computing device 200 may also include a communication interface 252 that connects to a network 253 for communicating with other computing devices.

Referring to FIGS. 1, 2, and 4-6 various methods 400-600 may be performed by way of associated computer-executable instructions according to some example aspects of the disclosure. In various example aspects of the disclosure, a memory device system (e.g., memory device system 130) is communicatively connected to a data processing device system (e.g., data processing device systems 110, otherwise stated herein as “e.g., 110”) and stores a program executable by the data processing device system to cause the data processing device system to execute various aspects of methods 400-600 via interaction with at least, for example, various databases. In these various aspects of the disclosure, the program may include instructions configured to perform, or cause to be performed, various ones of the instructions associated with execution of various aspects of methods 400-600. In some aspects of the disclosure, methods 400-600 may include a subset of the associated blocks or additional blocks than those shown in FIGS. 4-6. In some aspects of the disclosure, methods 400-600 may include a different sequence indicated between various ones of the associated blocks shown in FIGS. 4-6.

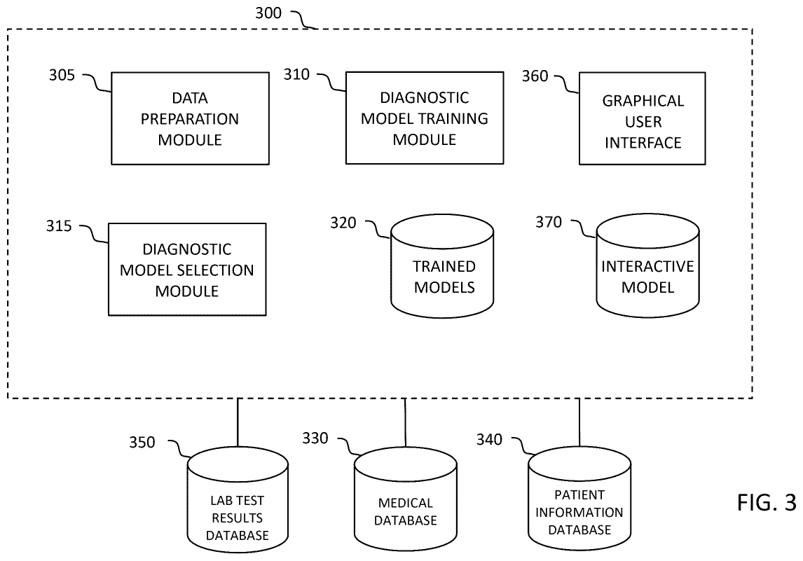

FIG. 3 shows an example of a diagnostic system 300 for assessing the risk of Addison's disease, according to some aspects of the disclosure. The system 300 may be a particular implementation of the systems 100, 200 according to some aspects. In some aspects of the disclosure, the diagnostic system 300 is implemented by programmed instructions stored in one or more memories and executed by one or more processors of the systems 100, 200.

In some aspects of the disclosure, the diagnostic system 300 includes a data preparation module 305, a diagnostic model training module 310, a diagnostic model validation/selection module 315, and one or more diagnostic models 320. In some aspects of the disclosure, the diagnostic system 300 may be communicatively connected to one or more databases 330, 340, and 350. In some aspects of the disclosure, the diagnostic system 300 includes a graphical user interface 360 to permit a user to interact with the system 300. In some aspects of the disclosure, the diagnostic system 300 includes a knowledge based third diagnostic model 370 which interacts with the user via the graphical user interface 360 to provide advanced guidance and diagnosis information. In some aspects of the disclosure, the one or more diagnostic models 320 may be stored in a database.

In some aspects of the disclosure, a medical database 330 stores reference medical data such as ranges of normal, low, and high test results for various diagnostic tests performed in the veterinary clinic or at a veterinary reference laboratory using various diagnostic testing instruments. In some aspects of the disclosure, the diagnostic tests may be performed by mobile laboratories, using home testing kits, etc. In some aspects of the disclosure, the diagnostic system 300 may access the medical database 330 to compare actual patient test results, stored in a laboratory test results database 350, with the typical ranges stored in the medical database 330 to interpret the test results performed at the veterinary clinic, the veterinary reference laboratories or using other means. In some aspects of the disclosure, the diagnostic tests include complete blood count (CBC), blood chemistry, PCR assays etc.

In some aspects of the disclosure, a patient information database 340 stores a patient's medical record, including patient demographic information, vital signs at each clinical visit, diagnoses, medications, treatment plans, progress notes, patient problems, vaccine history, test results, and imaging data such as radiographs. The demographic data may include species, breed, weight, age, gender, and geographic location, for example. In some aspects of the disclosure, the medical record may also include information on test results (for example, CBC, blood chemistry, pathology, urinalysis, serology, and PCR (polymerase chain reaction) panels/assays), vector of exposure, and diagnoses, obtained from the laboratory test results database 350.

In some aspects of the disclosure, the diagnostic system 300 is deployed on a point of care (POC) terminal located at a veterinarian's office or a clinic. The POC terminal may be connected to the veterinary reference laboratories or the diagnostic testing instruments at the veterinary clinic to receive test results for a patient. In some aspects of the disclosure, the POC terminal may be connected to one or more software servers. In some aspects of the disclosure, the software servers provide centralized software development resources and support for generating machine learning models (diagnostic models 320 and 370) for assessing the risk of Addison's disease. In some aspects of the disclosure, the diagnostic models 320 and 370 may be deployed and executed locally on the POC terminal. In other aspects of the disclosure, the diagnostic system 300 may be deployed on the server, with the POC terminal acting as a “client” that connects to the server, which executes the diagnostic models 320 and 370 based on patient information transmitted to the server from the POC terminal.

In some aspects of the disclosure, the diagnostic models 320 are used to provide alerts to clinicians when a patient has an increased likelihood of Addison's disease. In some aspects of the disclosure, further targeted screening may be performed using diagnostic model 370, in response to the alert, to validate the presence/absence of Addison's disease. This approach provides significant advantages for recognizing and treating Addison's disease by reducing the number of missed diagnoses of early-stage Addison's disease prior to an Addisonian crisis. Additional potential benefits of this approach include training veterinarians to recognize patients with Addison's disease prior to the development of classic electrolyte changes, promoting the value of including a resting cortisol test as part of the work-up of patients with gastrointestinal signs, and highlighting the potential significance of nonspecific recurring clinical signs in recognizing the risk for Addison's disease. This approach also identifies patients who may benefit from a resting cortisol test prior to elective procedures.

Generating machine learning models to assess the risk of Addison's disease poses several challenges. Conventional machine learning models work best when there is a large amount of training data with uniform distribution of positive and negative examples available, and the patterns that suggest the presence (positive) or absence (negative) of a particular disease are well-defined and separable from each other in the feature space. Addison's disease is relatively rare and can vary greatly in its presentation. Thus, the training set for the diagnostic model for Addison's disease is highly skewed, with much fewer examples of positive data samples then negative data samples. Moreover, the patterns (in feature space) for positive data samples (from patients with Addison's disease) are not well-defined and vary greatly, which makes it difficult to separate the clusters of positive samples from the clusters of negative samples (intermingling of positive and negative samples in the feature space) when training the diagnostic models 320 and 370.

To address these difficulties, a multi-stage approach is used to generate the diagnostic models 320. In some aspects of the disclosure, the diagnostic models 320 for assessing the risk for Addison's disease are generated using supervised machine learning method. The supervised method involves utilizing very large sets of anonymized patient data to provide a labeled training set of defined positive cases (patients who have the attribute of interest) and negative cases (patients without the attribute of interest). In some aspects of the disclosure, criteria for inclusion or exclusion of data samples in the training data set, and the definitions of positive and negative data samples, are defined by human medical experts. The computing device system 200, when presented with the training set, looks backwards at patient data samples in the months prior to the diagnosis of Addison's disease. From this data, the computing device system 200 uses mathematical algorithms to learn the patterns and relationships of weighted analytes and trends, along with other patient data, such as signalment, until one or more diagnostic models 320 that best predict the eventual diagnosis of Addison's disease are generated. Once the diagnostic models 320 are generated, they go through a preclinical technical validation for performance requirements on labeled data followed by a clinical evaluation in the field on unlabeled patient results before deployment.

In some aspects of the disclosure, a training data set with a wide patient population showing diverse clinical presentations is collected. This permits the diagnostic models 320 to encode a more realistic representation of positive cases seen in general practice. The collected data set is filtered to remove data samples that represent outliers or iatrogenic cases (where medications, surgery, or radiation induced Addison's disease) that could improperly skew the diagnostic models 320. For example, data samples from patients who are already taking medications for Cushing's or Addison's disease, and patients who have received corticosteroids or other medications are removed from the training set, to ensure that the training set represents “natural” presentations of Addison's disease. Similarly, data samples obtained from patients after a positive diagnosis for Addison's disease, or after a resting cortisol or adrenocorticotropic hormone (ACTH) stimulation test, are removed because the patent may have been administered treatment that masks the natural indicators of Addison's after positive diagnosis or based on the result of these tests. Other exclusion criteria may include data samples from patients who have had a resting cortisol above normal in the preceding months, data samples from patients with a medical history of a post ACTH>22 ug/dL (607 nmol/L) in the previous year, data samples from patients with a medical history consistent with being tested for Cushing's disease in the last year.

In some aspects of the disclosure, the data preparation module 305 communicates with the one or more databases 330, 340, 350 to receive laboratory test data, and medical record and medical history data for a large population of patients, gathered over a period of time. In some aspects of the disclosure, the medical record includes biographical information, such as age, gender, breed, and geographic location, one or more of which may be used as input features in training the machine learning models. These features can provide contextual discriminatory power to the machine learning models. In addition to biographical information, the medical record and the medical history data include information on any diagnostic tests that have been performed, and patient notes entered by a veterinarian. For example, a complete blood count (CBC) test includes data (results) for red blood cell count, hematocrit, hemoglobin, mean cell volume, mean corpuscular hemoglobin, mean corpuscular hemoglobin concentration, red blood cell redistribution width, reticulocytes (percentage and number), reticulocyte hemoglobin, nucleated red blood cells, white blood cell count, neutrophils (percentage and number), lymphocytes (percentage and number), monocytes (percentage and number), eosinophils (percentage and number), basophils (percentage and number), band neutrophils, platelet count, platelet distribution width, mean platelet volume, plateletcrit, total nucleated cell count, agranulocytes (percentage and number), and granulocytes (percentage and number). Other tests may check for presence, count, or concentration of squamous epithelial cells, non squamous epithelial cells, bacteria (rods or cocci), hyaline and nonhyaline casts, or crystals (bilirubin, ammonium biurate, struvite etc.). Levels of various proteins, enzymes, or minerals may have been checked. Other tests to check for specific pathogens may have been run, and their results stored in the medical record and the medical history data. Information on the diagnosis of Addison's disease, and the date of the diagnosis may also be stored in the medical record and medical history data.

In some aspects of the disclosure, the data preparation module 305 receives the laboratory test data and the medical record and medical history data, and extracts features to be used as inputs for training machine learning models to classify Addison's disease. Ground truth (labeling of the data samples as positive or negative examples of Addison's disease) may be obtained from the diagnosis information, if expressly stored in the medical record and medical history data, or by using other criteria for labeling the data samples in the training data set. For example, in some aspects of the disclosure, positive Addison's disease cases may be defined as data samples from patients with ACTH stimulation results consistent with Addison's disease (for example, pre- and post-cortisol result <2 ug/dL (55 nmol/L) on a same day performed prior to administration of treatment for Addison's disease). The remaining data samples, which do not meet the criteria for positive data samples, are labeled as negative examples. In some aspects of the disclosure, the data preparation module 305 also filters the collected data samples based on predefined exclusion criteria to generate a labeled training dataset.

In some aspects of the disclosure, positive Addison's disease cases may be defined as samples associated with medical records including treatment protocols consistent with the treatment of Addison's disease. In examples, in response to identifying a risk of Addison's disease, intravenous fluids are administered, medication such as glucocorticoids and/or mineralocorticoids are administered, and/or diet changes including low-sodium/low-fat diets are prescribed.

In some aspects of the disclosure, the data preparation module 305 determines the ratio of positive and negative labeled data samples in the training dataset and uses data sampling techniques to achieve a more balanced training dataset. For example, if the training data set has 10 times as many negative labeled examples as positive labeled examples, the data preparation module 305 may sample the negative examples to select 1 out of every 5 negative examples, to achieve a dataset that has twice as many negative labeled data samples as positive labeled data samples. It is readily understood that any predefined ratio for negatively labeled data samples to positively labeled data samples may be used, and the above example is provided merely for purposes of illustration only.

Data sampling techniques include random sampling, stratified sampling, cluster sampling, importance sampling, uncertainty sampling, and active learning, as examples. Random sampling entails randomly selecting the desired number of data samples from the larger pool of data samples. Random sampling is simple and cost effective, generally ensures that the selected subset is a representative sample of the overall data distribution, and can be implemented by randomly selecting a fixed number of instances or by specifying a percentage of the collected data samples to be included in the training set. Stratified sampling is useful when the class distribution of the selected data samples needs to be maintained. For example, in a case where the larger pool of collected data samples (negative examples) can be divided into different classes or categories, each class or category is proportionally represented in the selected data samples to ensure diversity of the data. Cluster sampling involves partitioning the data into clusters based on certain criteria (e.g., geographic location, demographics, or other relevant factors). Instead of sampling individual instances of patient data samples, each cluster is samples to ensure that a representative data set is collected. This can be beneficial when clusters represent distinct groups or subpopulations within the data, such as different breeds of animals. Importance sampling assigns weights to each data sample based on its importance or relevance to the diagnostic task. Data samples that are more informative or challenging can be given higher weights, while less informative samples receive lower weights. This technique allows prioritizing the inclusion of important data samples in the training dataset. Uncertainty sampling focuses on selecting data samples that the current model is uncertain about or finds challenging to classify. By selecting these data samples, the diagnostic system actively targets areas of the data where the trained machine learning model needs further improvement. Common uncertainty sampling strategies include selecting instances with the highest prediction entropy, margin, or confidence scores. Active learning is an iterative sampling approach where the trained machine learning model interacts with the data preparation module 305 to select the most informative data samples for further training. The model can query the diagnostic system for instances it is uncertain about or instances that are expected to have the most impact on improving its performance. This interactive process helps optimize the data selection based on the evolving needs of the machine learning models.

In some aspects of the disclosure, the data samples in the training set are represented using feature vectors that include various analytes such as electrolyte levels (sodium, potassium, chloride, sodium-to-potassium ratio), glucose, urea (blood urea nitrogen—BUN), creatinine, albumin, alkaline phosphatase (ALP), red blood cell count (RBC), monocytes, lymphocytes, &lymphocytes reticulocyte, % reticulocyte, neutrophils, % neutrophils, eosinophils, % eosinophils, age, cholesterol, amylase, and calcium. It should be understood that the features included in the data samples are not limited to those listed here, and this list is provided for purposes of illustration only. Other features could be obtained from the patient's medical record and medical history or various laboratory tests such as CBC, basic chemistry panels, or more extensive chemistry panels, etc.

The feature space for assessing the risk of Addison's disease is very complex because of the potential number of features and because not all patients will have available information for all features. Complex feature spaces pose challenges for machine learning for several reasons such as dimensionality, computational complexity, overfitting, sparsity etc. As the number of potential features (dimensions) that can be used for assessing the risk of Addison's disease increases, the volume of the feature space grows exponentially. This phenomenon is known as the curse of dimensionality. In high-dimensional spaces, the available data becomes sparse, making it challenging for machine learning algorithms to generalize well. Models may struggle to find patterns or relationships when the number of features is much larger than the number of samples. With an increasing number of features, the computational cost of training and evaluating machine learning models tends to rise. Many algorithms have time and space complexity that scales with the number of dimensions, making them computationally expensive in high-dimensional spaces. High-dimensional spaces also increase the risk of overfitting, where models capture noise or specific characteristics of the training data that do not generalize well to new, unseen data. This is also a direct consequence of the sparsity of data in complex feature spaces. Models may fit the training data too closely, capturing spurious correlations or outliers, rather than learning the underlying patterns because the available data points may be sparsely distributed. This sparsity makes it difficult for machine learning models to identify meaningful relationships between features and the target variable. Insufficient (sparse) data can also lead to poor model performance and unreliable predictions.

Some machine learning algorithms, especially those with a large number of tunable parameters, may become more difficult to train and fine-tune in high-dimensional spaces. This can lead to challenges in finding the right machine learning models for use in assessing the risk of Addison's disease. Moreover, some algorithms may become numerically unstable in high-dimensional spaces, leading to issues such as a lack of convergence during training or difficulties in optimizing the model parameters. In some aspects of the disclosure, the complexity of the feature space is reduced by performing feature selection-identifying only the most relevant/discriminative features and removing other features that do not significantly boost the performance of the machine learning models. However, it can be difficult to distinguish between informative and irrelevant features, leading to suboptimal model performance. To address these challenges, feature engineering, dimensionality reduction techniques (e.g., principal component analysis), and careful model selection are crucial. Ensemble learning, which combines multiple models, can be effective in mitigating the impact of complex feature spaces by leveraging the strengths of different models.

In some aspects of the disclosure, ensemble learning is used to generate a plurality of machine learning diagnostic models 320 (ensemble of models 320), which are trained using the same or different sets of features. This approach permits the diagnostic system 300 to optimize different diagnostic models 320 on different portions of the sample space, and is especially useful for assessing the risk of Addison's disease due to its varied presentations. The outputs from the various machine learning diagnostic models 320 are merged or combined using ensemble methods to produce a single model/output that encompasses the strengths and compensates for the weaknesses of each individual diagnostic model 320.

In some aspects of the disclosure, the machine learning model is generated by performing data collection, data splitting, model selection, model training, and model validation. Data collection has been discussed in detail above and entails gathering a representative data set that is relevant to assessing the risk of Addison's disease. Since bias and inconsistencies within the data can significantly impact a diagnostic model's performance, thorough data cleaning and pre-processing are performed. This includes addressing missing values in the data samples, identifying and correcting inconsistencies, and transforming the data into a format suitable for the selected type of machine learning model. In some aspects of the disclosure, feature selection is performed to identify the most discriminative features and, potentially, reduce the complexity of the machine learning model. Once the data set is ready, it is divided into at least two separate training and test sets. In some cases, a separate validation set may also be generated. The training set serves as the labeled ground truth for training the diagnostic models 320 to learn the underlying patterns and relationships within the data. The test set is not presented to the models during training and is used to assess the models' ability to generalize and predict accurately on new data. The validation set is used during training to help the models generalize and avoid overfitting. During training, the machine learning algorithm iteratively adjusts the internal parameters of each diagnostic model 320 to minimize prediction errors on the training set. This process, known as optimization, aims to ensure that the diagnostic models 320 learn the optimal representations and decision boundaries for accurately classifying the data samples. The choice of appropriate hyperparameters, which control the learning process, plays a crucial role in achieving optimal performance. Hyperparameters govern the learning process and significantly impact the diagnostic model's performance. Tuning these parameters involves finding the optimal settings that maximize model accuracy and generalization. In some aspects of the disclosure, techniques like grid search and random search are employed to systematically explore the parameter space and identify the optimal configuration. Once the diagnostic models 320 are trained, their performance is evaluated using the test set. This involves measuring the models' accuracy, precision, recall, and other relevant metrics. These metrics provide valuable insights into the models' strengths and weaknesses, allowing for further refinement and improvement. Cross-validation, which involves evaluating the models on multiple splits of the data, provides a more robust estimate of their generalization ability. Both hyperparameter tuning and cross validation can be performed during either of the training or the testing phases, to improve the models' performance.

Ensemble learning is an advanced machine learning technique that combines predictions from multiple diagnostic models 320 to produce a more accurate and robust final prediction. This approach leverages the diversity of individual diagnostic models 320 to mitigate biases and further improve overall performance, making it particularly effective in complex and varied datasets. The idea behind ensemble learning is that by combining diverse independent models, the weaknesses of one model can be compensated for by the strengths of others, and the aggregation of the models result in a more accurate and reliable prediction. Diversity means that the different machine learning models within the ensemble make different types of errors. This diversity ensures that the ensemble of models 320 can collectively address a broader range of scenarios. The models within the ensemble are designed to be as independent as possible to reduce the risk of systematic errors, because similar models might make the same mistakes. The predictions of individual models 320 are combined through a predefined aggregation strategy, such as averaging, voting, or weighted voting. The goal is to leverage the collective intelligence of the ensemble.

The machine learning models in the ensemble of models 320 can be homogeneous, heterogeneous, or a mix of both. Homogeneous models refer to using more than one machine learning model of the same type, but with intentional variations. For instance, in the case of a decision tree machine learning model, different decision trees having different depths, minimum samples per leaf, or feature subsets maybe generated during training. Heterogeneous models involve combining different types of models, each contributing unique perspectives. This could include combining decision trees with support vector machines, k-nearest neighbors, or neural networks, creating a diverse set of predictors. In some aspects of the disclosure, one or more ensemble learning training methods are used to train the ensemble of diagnostic models 320. The ensemble learning training methods include bagging, boosting, and stacking. Bagging, also known as bootstrap aggregating, involves generating multiple instances of a same type of machine learning model on different subsets of the training data. Each subset is obtained through bootstrapping (random sampling with replacement or another data sampling technique discussed above). Each instance of the machine learning model is trained independently using one subset of the training data. This process introduces diversity by exposing each model to slightly different instances and reduces overfitting. Different operators, such as the average (for regression) or majority vote (for classification) of the individual model predictions, are used for the final prediction. In some aspects of the disclosure,

Boosting entails building a sequence of diagnostic models, where each subsequent diagnostic model in the sequence tries to reduce the errors of the preceding diagnostic model in the sequence. Boosting assigns weights to instances based on their performance in earlier iterations, emphasizing the importance of misclassified data points. Higher weights are assigned to misclassified instances and lower weights to correctly classified ones and a weak learner is trained on the weighted dataset. The model's performance is evaluated using the test data set and the errors are used to adjust weights. This process is repeated iteratively, giving more emphasis to misclassified instances in each iteration, until the error in prediction is below a desired threshold. Algorithms like AdaBoost and Gradient Boosting exemplify this approach, refining the model iteratively to enhance overall predictive performance.

Stacking involves training multiple different types of machine learning models using the techniques discussed above, and then combining their predictions using another model called a meta-model or blender. The meta-model learns to weigh the predictions of the individual models to create a final prediction.

To ensure robustness and generalization of the trained diagnostic models 320 in the ensemble, both technical (cross-fold) and real-world (clinical trial) validation is employed. This rigorous validation process helps prevent overfitting and provides a more accurate estimate of the models' performance. The predictions from each of the plurality of diagnostic models in the ensemble maybe combined using different functions to generate a single final prediction. For example, in hard voting, the final prediction is determined by the majority vote of the diagnostic models 320. Each diagnostic model has equal weight in the decision-making process. As another example, soft voting considers the confidence or probability assigned to each class by each diagnostic model 320. The final prediction is a weighted combination of these probabilities, providing a more nuanced decision. As yet another example, weighted averaging may be used to assign a weight to each model's prediction based on the model's performance during validation (technical and/or clinical trial). In weighted averaging, diagnostic models with higher accuracy or reliability are given more significant influence in the final prediction, allowing for a dynamic weighting scheme.

FIG. 4 shows a flowchart of an ensemble learning based Addison's disease risk assessment method 400, according to various aspects of the disclosure. In step 410, the data preparation module 305 prepares the data sets for training, testing, and validating the diagnostic models 320 for the diagnostic system 300. The raw data samples are obtained from diagnostic tests performed either at laboratories or using diagnostic instruments at the POC terminal, and clinical history data derived from integrated veterinary clinic practice information management software (PIMS). In some aspects of the disclosure, the data samples are collected over a period of time and stored in the one or more databases 330, 340, and 350, discussed above. Ground truth for the data samples is defined as ACTH stimulation test results consistent with Addison's disease. A wide patient population with diverse clinical presentations is included in the training data set to provide the diagnostic models 320 with a more realistic representation of the general patient population. Since the goal of the diagnostic system 300 is to detect early or preclinical cases in addition to more classic presentations, the positively labeled data samples are not restricted to those with clinical signs or to patients in whom Addison's disease is already suspected. In some aspects of the disclosure, medical experts defined an Addison's phenotype to label positive data samples (i.e., patients with Addison's disease) and negative data samples (i.e., patients without Addison's disease). The phenotype involves several inclusion and exclusion criteria to label data samples as positive or negative. For example, positive data samples include adult patients with an ACTH stimulation test consistent with hypoadrenocorticism (pre- and post-ACTH stimulation cortisol results <2 μg/dL [55 nmol/L]), without recent systemic or topical glucocorticoid treatment, and with no history of prior diagnosis of or treatment for Cushing's disease. Patients under or over predefined ages (such as dogs <1 year old or >15 years old) are excluded because data samples from very young or very old patients are misleading and tend to be outliers that are not representative of the markers for Addison's disease. Addisonian patients with and without electrolyte abnormalities are included to enable the ensemble machine learning model 320 to be able to identify both typical and atypical Addison's cases. In some aspects of the disclosure, the collected data samples are divided into a plurality of data subsets for training, validating, and testing the diagnostic models.

The data samples in the training, validating, and testing data sets include features such as complete blood count and serum chemistries (e.g., blood urea nitrogen [BUN], sodium, and potassium) as well as demographic data like gender, breed, and age. The minimum requirements for the diagnostic models include commonly assessed parameters as found in CBC and basic chemistry panels with electrolytes. For enhanced detection of atypical or early Addison's dogs, a more extensive chemistry panel is recommended due to the inclusion of other helpful analytes such as cholesterol. The model parameters are optimized to meet predetermined performance criteria of specificity of ≥95% with sensitivity of at least 50%.

In step 420, the data set is filtered by one or more criteria discussed above to extract the relevant set of data samples for training, validating, and testing the machine learning models 320. In step 430, the diagnostic model training module 310 selects and trains an ensemble of diagnostic models 320 using the ensemble learning methods discussed previously. In some aspects of the disclosure, in step 430, a plurality of decision trees (random forest of trees) is trained on different feature subsets in the data samples to generate the ensemble of diagnostic models 320. A decision tree is a machine learning model with a tree-like structure where each internal node represents a feature (or attribute) of the data set, and each branch represents a possible outcome of the feature. At each node, the algorithm selects the best feature based on a splitting criterion, such as information gain or Gini impurity. This process continues recursively until reaching a leaf node, which represents the final predicted class or value. Decision trees are easy to interpret and understand, and can be generated using expert domain knowledge (a rule based approach). The tree structure provides a clear visual representation of the decision-making process. Moreover, decisions trees are robust to irrelevant features because the algorithm automatically ignores features that are not relevant for prediction. However, decision trees are prone to overfitting and can become too complex in a large feature space, leading to poor performance on unseen data. Decision trees are also sensitive to noise in the data; slight changes in the data can lead to significant changes in the tree structure. Each tree in a random forest is trained independently and makes its own prediction. The final prediction is then obtained by taking the majority vote from all trees. This approach is more robust to outliers and noise in the data.

Random forests are ensemble learning algorithms that combine multiple decision trees to improve overall performance. They work by building a forest of individual decision trees, each trained on a different subset of the data and using a random subset of features at each split. The predictions from these individual trees are then combined to make the final prediction. Random forests provide improved accuracy and robustness over individual decision trees. By combining multiple decision trees, random forests are less prone to overfitting and more robust to noise in the data, and can handle complex relationships between features. Random forests can learn complex relationships between features that may not be easily captured by a single decision tree. Although more computationally expensive to train, and less interpretable than individual decision trees, the random forest ensemble models outperform individual decision trees because assessing the risk of Addison's disease is a complex problem with many features and intricate relationships.

In some aspects of the disclosure, in step 430, a gradient boosting approach is used to build a sequence of decision trees using ensemble learning. Gradient boosting is a powerful technique for building ensemble models by sequentially adding weak machine learning models (models with low individual accuracy) to improve overall performance. The training process starts with an initial model, often a simple model like a single decision tree. The algorithm calculates the pseudo-residuals, which represent the difference between the actual and predicted values for each data sample, and iteratively adds weak machine learning models. In each iteration, a new weak machine learning model is trained on the current pseudo-residuals. This new machine learning model focuses on correcting the errors made by the previous machine learning models. The learning rate determines the weight given to each new machine learning model in the ensemble. The (weighted) predictions of all weak machine learning models are combined to generate the final prediction at each iteration. The iteratively process of adding weak machine learning models and updating predictions continues until a stopping criterion is met. Common stopping criteria include reaching a desired level of accuracy or exceeding a maximum number of iterations. Some benefits of using gradient boosting to build an ensemble model include improved accuracy, reduced variance, flexibility, and scalability. By combining the predictions of multiple weak machine learning models, gradient boosting can achieve significantly higher accuracy than individual learners. Predictions are made by majority vote of the weak machine learning models; predictions, weighted by their individual accuracy. Further, since gradient boosting focuses on correcting errors made by previous machine learning models, leading to a more robust ensemble model with lower variance. Gradient boosting can be used with various types of machine learning models, such as decision trees, linear regression models, and others, making it easy to generate heterogenous models for the ensemble of diagnostic models.

In steps 440 and 450, the diagnostic model selection module 315 performs technical and clinical validation of the trained ensemble of diagnostic models 320, respectively. Technical validation, performed in step 440, ensures that the diagnostic models 320 meet the performance requirements, are unbiased, and are strong enough to support a clinical evaluation prior to deployment. The preclinical technical validation is performed by running the model against the validation and test data sets of cases. In some aspects of the disclosure, the training set is used to train the ensemble model, the validation set is used to tune hyperparameters of the ensemble model and monitor the model for overfitting during training. The test set is used to evaluate the final model's performance on unseen data. The machine learning models used in ensemble learning (whether random forest, gradient boosting models, or other machine learning models) have several hyperparameters that can significantly impact their performance. Techniques like grid search or random search can be used to identify the optimal values of these hyperparameters. The validation data set is essential for evaluating the performance of different hyperparameter configurations and selecting the best ones for generating the structure of the machine learning models included in the ensemble. In some aspects of the disclosure, metrics such as loss function, accuracy, and AUC can be monitored during model training and validation to improve the model's performance.

In some aspects of the disclosure, a loss function measures the difference between the predicted and actual values for a data sample. Lower values of the loss function indicate better model performance. Common loss functions include mean squared error (MSE), which measures the average squared difference between predicted and actual values, cross-entropy, which measures the difference between the predicted probability and the actual class, and logarithmic loss, which is like cross-entropy but more sensitive to misclassified examples. Accuracy represents the percentage of predictions that are correct. It is a simple and intuitive metric but can be misleading when dealing with imbalanced datasets. AUC (area under the curve) measures the ability of a classifier to distinguish between classes. The AUC represents the area under the receiver operating characteristic (ROC) curve, which plots the true positive rate (TPR) against the false positive rate (FPR) at different thresholds. An AUC of 1 represents a perfect classifier, while an AUC of 0.5 represents a random guess. In some aspects of the disclosure, one or more of the loss function, accuracy, and AUC are observed. If these metrics plateau or worsen on the validation data set, the trained ensemble model likely suffers from overfitting. In this case, early stopping techniques are used to stop training once the validation metrics start declining, preventing overfitting.

In some aspects of the disclosure, cross validation is used to monitor the ensemble diagnostic model 320 for overfitting. Cross-validation is a resampling procedure used to evaluate machine learning models on a limited data sample. The procedure has a single parameter called k that refers to the number of groups that a given data set is to be split into. As such, the procedure is often called k-fold cross-validation. When a specific value for k is chosen, such as k=10, the procedure becomes 10-fold cross-validation. Cross-validation is primarily used to estimate how the trained model is expected to perform in general when used to make predictions on data not used during the training of the models 320. The dataset is shuffled randomly and divided into a predefined number (k) of groups. The training and validation process is performed k times, with one of the groups of data being held out as the validation set for each iteration and the remaining k-l groups being used as the training set. Each model is fitted (trained) on the training set and evaluated (validated) on the test set to determine the level of generalization of the trained models. The purpose of k-fold cross validation is not to pick one of the trained models 320 as the final machine learning model but, rather, to help determine the model structure and the hyperparameter tuning process for each machine learning model 320. Once the ensemble model 320 is trained and hyperparameters are tuned, its performance is evaluated using the test data set.

In step 450, a clinical validation of the ensemble of diagnostic models 320 is performed to measure the model's effectiveness in assessing the risk for Addison's disease, prior to deployment (step s460). In some aspects of the disclosure, the clinical validation is performed by deploying the diagnostic models 320 on a test basis at a selected number of clinics, evaluating new data samples from patients using the trained diagnostic models 320, and validating the prediction of the diagnostic models 320 using expert opinion of the veterinarians. Further diagnostic tests may be run, in the case where the ensemble model 320 predicts a higher risk of Addison's disease, to determine whether the patient is suffering from Addison's disease.

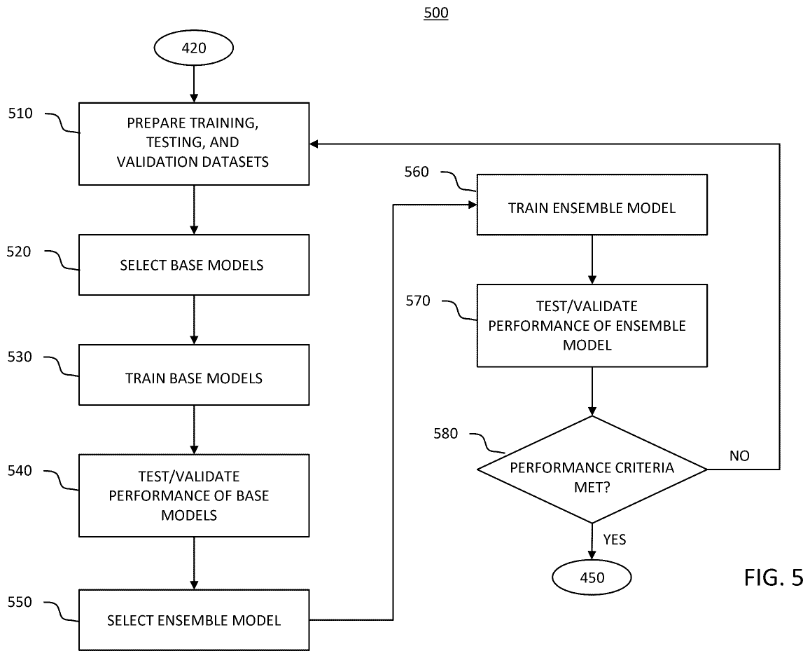

FIG. 5 shows a flowchart with additional details for a method 500 of training the ensemble of diagnostic models 320, according to some aspects of the disclosure. In step 510, the collected and filtered medical data (from steps 410 and 420) is pre-processed and split into training, testing, and validation datasets using the sampling techniques discussed above. In step 520, the individual base models (learners) for the ensemble of models are selected. The learners may include one or more machine learning models such as decision trees, random forests, support vector machines, neural networks etc. A diverse set of learners may be selected for better ensemble performance. Multiple instances of a same type of machine learning model with different architecture or parameter sets may also be selected. In step 530, each base model (learner) is trained independently on the training data set using model-specific training methods. In step 540, each base model is tested/validated for performance using technical validation methods such as cross-validation. The training and validating steps 530 and 540 are repeated iteratively until predefined performance criteria for each base model is met. Performance metrics (e.g., accuracy, loss) on training and testing/validation data are measured and tracked to improve the performance of each base model. In some aspects of the disclosure, early stopping is used to prevent overfitting during the training step 530 based on the performance metrics. During the training and validating steps 530 and 540, the hyperparameters (e.g., tree depth, number of estimators etc.) for each model are tuned.

In step 550, the ensemble architecture (e.g., bagging, boosting, stacking), the learning approach (e.g., parallel, sequential), and the prediction aggregation strategy (e.g. averaging, voting) is selected. In step 560, the ensemble of diagnostic models 320 is trained using the outputs or predictions of the base models. In some aspects of the disclosure, the base models may be weighted differently based on their performance, permitting models with better performance to have more influence on the output of the ensemble model 320. In step 570, the ensemble model's performance is validated on unseen validation data. The ensemble model's performance is also compared with individual base model performance to check for overfitting (high training accuracy, low validation accuracy). In some aspects of the disclosure, in step 580, results from the ensemble model 320 are analyzed. If the performance meets the desired criteria, the ensemble model is ready for clinical validation and deployment. If the performance does not meet the desired criteria, areas for improvement are identified and steps 510-570 are repeated to adjust the base models, ensemble architecture, hyperparameters, or data pre-processing as needed. Training and validation of the individual learners and the ensemble model are repeated until the desired performance is achieved.

In some aspects of the disclosure, the diagnostic models 320 (the ensemble of diagnostic models) include at least a first machine learning diagnostic model that is trained on electrolyte abnormalities/features and a second machine learning diagnostic model that is not trained on electrolyte abnormalities/features. In some aspects of the disclosure, the diagnostic system 300 includes a graphical user interface 360 that permits a user to further engage with an interactive diagnostic model 370 when any of the diagnostic models in the ensemble of models 320 indicate a risk of Addison's disease. In these aspects of the disclosure, the ensemble of diagnostic models 320, discussed above, provides an indication that the veterinarian should perform additional screening to assess the risk for Addison's disease.

In some aspects of the disclosure, the additional screening is performed in an interactive manner by a user (for example, the veterinarian) engaging with the interactive diagnostic model 370 via the user interface 360. The user interface presents a sequence of questions, dynamically selected by the third diagnostic model 370 based on answers provided to previous questions, to guide the veterinarian towards additional testing to confirm the presence or absence of Addison's disease.

For example, if either the first diagnostic model or the second diagnostic model in the ensemble of models 320 indicate that the patient may have Addison's disease, the third diagnostic model 370 may first query the veterinarian to provide additional information on recent medications taken by the patient. If the patient has recently been treated for Cushing's disease, or has recently been prescribed steroids, then the indication for Addison's from one of the plurality of diagnostic models 320 is likely a false positive and the third diagnostic model 370 may provide indications for the veterinarian to monitor the patient during treatment and recheck for Addison's after the current treatment is completed. If the patient is not taking medications that cause the patient's bloodwork to have similar patterns as those for Addison's disease, then the third diagnostic model 370 may further query the veterinarian for additional information on clinical presentation and patient history. This information is utilized along with results of recent tests (such as electrolyte panels, ACTH stimulation test, dexamethasone suppression test etc.) to train the third diagnostic model 370 to provide further diagnostic guidance and evaluation.

In some aspects of the disclosure, the third diagnostic model 370 includes a knowledge based expert system that combines both contextual domain knowledge and data-driven training to improve diagnostic accuracy and efficiency. In some aspects of the disclosure, the training phase of the third diagnostic model 370 includes generating a knowledge base that encodes expert knowledge in diagnosing Addison's disease. The expert knowledge is obtained by interviewing medical professionals specializing in Addison's disease to capture their diagnostic reasoning, differential diagnoses, and treatment strategies. The expert knowledge may also be gathered by analyzing (using either human experts or machine learning models) medical textbooks, research papers, documented cases (data) and guidelines for evidence-based practice. The acquired knowledge is appropriately structured and encoded for the training to be performed efficiently. For example, in some aspects of the disclosure, the knowledge about relationships between symptoms, findings, and diagnoses can be captured using production rules such as “IF-THEN” statements. In other aspects of the disclosure, the knowledge may be encoded as a Bayesian networks, which represents causal relationships between factors using probability. This permits the third diagnostic model 370 to consider the likelihood of different diagnoses based on various combinations of symptoms and test results. In yet other aspects of the disclosure, a semantic network may be used to connect symptoms, diseases, and tests through directed links, creating a web of interconnected knowledge that facilitates efficient information retrieval and inference.

In some aspects of the disclosure, the expert knowledge defines general rules and guidelines for the knowledge base, which are then refined using patient medical history and test data (such as electrolyte panels, ACTH stimulation test, dexamethasone suppression test, etc.) to train the third diagnostic model 370 to provide further diagnostic guidance and evaluation. The data is collected from diverse data sources, including electronic medical records, laboratory test results, clinical signs, and medication histories. The data is cleaned and validated, any missing or inconsistent values are addressed, and potential biases are removed before using the data for training. Data mining techniques like association rule mining and clustering are employed to identify hidden patterns and correlations within the data. This data mining analysis uncovers associations between specific symptom combinations and diagnoses, or reveal risk factors and disease progression patterns. The patterns and insights extracted using data mining are then used to refine the existing rules in the knowledge base. The data mining techniques used for training the third diagnostic model 370 automatically discover previously unknown relationships between symptoms and diagnoses, not readily apparent from expert knowledge alone. This allows the third diagnostic model 370 to stay relevant and adapt to evolving disease patterns and emerging medical literature.

In some aspects of the disclosure, the third diagnostic model 370 integrates probabilistic reasoning techniques with a rules-based engine to account for data variability and incomplete information. This enables the diagnostic system 300 to provide confidence levels associated with diagnoses, enhancing transparency and guiding further investigative steps. The third diagnostic model 370 employs an inference engine that translates the knowledge base and the responses to the queries from the user interface 360 into actionable insights. In some aspects of the disclosure, the inference engine includes both forward chaining and backward chaining inference strategies.

Forward chaining starts with known facts (symptoms, test results) and applies the rules iteratively to infer additional facts and ultimately reach a diagnosis/conclusion. Backward chaining works backward from a suspected diagnosis/conclusion, identifying supporting evidence and tests needed to confirm or refute it. Backwards chaining is especially useful to identify additional information that is needed to support a diagnosis or conclusion, and query the veterinarian, using the graphical user interface 360, to provide the additional information. The user interface 360 of the diagnostic system 300 permits the veterinarian to not only input patient symptoms but to also assign relative weights to their severity or duration, providing the diagnostic system 300 with richer context.

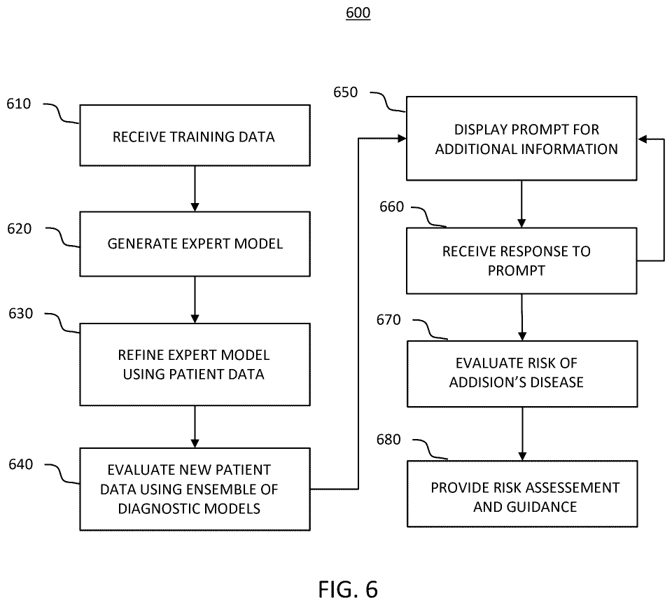

FIG. 6 shows a flowchart of a method 600 for training and using an interactive knowledge based model for assessing Addison's disease risk, according to various aspects of the disclosure. In step 610, data for training the knowledge based model is obtained. The training data includes both domain knowledge and clinical/laboratory patient data. The domain knowledge is obtained, for example, from human experts. In step 620, an expert model is generated using the obtained domain knowledge. The expert model may include rules-based inference, knowledge graphs, semantic ontologies, predicate logic or other techniques, known in the art, for representing domain knowledge. In step 630, the expert model is refined using the clinical/laboratory patient data. This refinement could include adjusting the logic defined in the expert model, assigning weights to the various logic branches, adjusting the connections between various entities in the model etc.

In step 640, new patient data is received and assessed using the ensemble of diagnostic models 320. If the ensemble of diagnostic models indicates a potential risk of Addison's disease, in steps 650 and 660, an interactive user interface is used to present a clinician with a sequence of prompts and receive responses to the sequence of prompts, to acquire additional data to assess the risk of Addison's disease. Each new prompt presented in step 650 may be based on the response received to a previous prompt in step 660. Steps 650 and 660 may be repeated, as appropriate, to acquire the additional information required by the knowledge based model. In step 670, the risk of Addison's disease is determined based on the acquired additional data. In step 680, the user interface is used to provide the clinician with an assessment of the risk for Addison's disease on the new patient data and guidance for further testing and treatment.

In some aspects of the disclosure, the diagnostic system 300 provides differential diagnosis suggestions, presenting a list of possible diagnoses based on the entered information, considering the patient's medical history and test results. In some aspects of the disclosure, the diagnostic system 300 provides evidence-based recommendations, suggesting further tests or investigations based on the third diagnostic model's 370 assessment and current clinical guidelines. In some aspects of the disclosure, the diagnostic system 300 provides explanatory reasoning for the diagnostic model's conclusions and guidance, showing the reasoning chain behind the suggested diagnoses and tests, promoting transparency and trust with the veterinarian.

In some aspects of the disclosure, the diagnostic system 300, including the ensemble of diagnostic models 320 and the third diagnostic model 370, is continually evaluated and refined using anonymized patient data to assess its accuracy in clinical settings. Validation of the diagnostic system 300 is also performed based on feedback from medical professionals using the diagnostic system 300, to identify areas for improvement and ensure its practical utility. In some aspects of the disclosure, the knowledge base is regularly updated to incorporate new medical knowledge from medical experts, research, guidelines updates, and emerging disease patterns.

In some aspects of the disclosure, the assessed risk of Addison's disease is compared against a predefined threshold, and/or other indications (symptoms and clinical presentations of the patient) to determine whether to administer one or more treatments for Addison's disease. In some aspects of the disclosure, in response to the assessed/identified risk of Addison's disease being greater than the predefine threshold, and/or based on the other indications, intravenous fluids are administered, medication such as glucocorticoids and/or mineralocorticoids are administered, and/or diet changes including low-sodium/low-fat diets are prescribed.

Various aspects of the diagnostic system 300 may be realized by software, or more precisely, an application program running on a microprocessor, or by firmware or hardware implementing the program on the POC terminal and/or the software servers POC. The diagnostic system 300 may include one or more memories which store various data and program modules associated with the diagnostic methods. It is important to note that the machine learning and diagnostic algorithms described herein do not represent a computer application of the way humans perform diagnoses. Humans interpret new data in the context of everything else they have previously learned. In stark contrast to mental diagnostic processes, artificial intelligence algorithms, and specifically, the machine learning (ML) algorithms described herein, analyze massive data sets to identify patterns and correlations, without understanding any of the data they are processing. This process is fundamentally different from the mental process performed by a veterinarian. Furthermore, the large amounts of data required to the train the machine learning models, and the complexity of the trained models, make it impossible for the algorithms described herein to be performed merely in the human mind.

Accordingly, it should now be understood that concepts of the present disclosure are directed to ensemble learning methods and systems for selecting and training an ensemble of diagnostic models for assessing the risk of Addison's disease in patients.

Numbered aspects of the present disclosure are provided below:

In a first aspect A1, the present disclosure provides a method, executed by a programmed data processing device system, of receiving first medical training data; training a plurality of first machine learning models on the received first medical training data to generate an ensemble of diagnostic models; receiving second medical training data; training a second machine learning model on the received second medical training data to generate a knowledge based diagnostic model; receiving new patient data; determining whether the ensemble of diagnostic models indicates a risk for Addison's disease for the new patient data; and, in a case where the ensemble of diagnostic models indicates a risk for Addison's disease for the new patient data, assessing the risk of Addison's disease using the knowledge based diagnostic model.

In a second aspect A2, the present disclosure provides the method according to aspect A1, wherein the ensemble of diagnostic models includes one or more of a decision tree model, a random forest model, a neural network, a support vector machine, or a nearest neighbor classifier.

In a third aspect A3, the present disclosure provides the method according to any one of aspects A1-A2, wherein training the plurality of first machine learning models includes selecting at least a first machine learning model and a second machine learning model for the ensemble of diagnostic models, wherein the second machine learning model is different from the first machine learning model; training the first machine learning model using a first subset of the received first medical training data to generate a first model of the ensemble of diagnostic models; and training the second machine learning model using a second subset of the received first medical training data to generate a second model of the ensemble of diagnostic models, wherein the second subset of the received first medical training data is different from the first subset of the received first medical training data, and wherein the risk for Addison's disease for the new patient data is determined based on outputs of the first machine learning model and the second machine learning model.

In a fourth aspect A4, the present disclosure provides the method according to aspect A3, wherein it is determined that the ensemble of diagnostic models indicates a risk for Addison's disease for the new patient data in a case where the outputs of either the first machine learning model or the second machine learning indicate the risk for Addison's disease for the new patient data.

In a fifth aspect A5, the present disclosure provides the method according to any one of aspects A1-A4, wherein training the second machine learning model includes generating an expert model including a plurality of rules based on domain knowledge included in the received second medical training data; refining the plurality of rules in the expert model based on patient data included in the received second medical training data; and generating the knowledge based diagnostic model based on the refined plurality of rules.

In a sixth aspect A6, the present disclosure provides the method according to any one of aspects A1-A5, wherein the knowledge based diagnostic model includes one or more of a rules based model and a data driven machine learning model.

In a seventh aspect A7, the present disclosure provides the method according to any one of aspects A1-A6, wherein assessing the risk of Addison's disease using the knowledge based diagnostic model further includes displaying, on a user interface, a series of prompts to receive further patient medical data; and assessing the risk of Addison's disease based the received further patient medical data in response to the series of prompts.

In an eighth aspect A8, the present disclosure provides the method according to aspect A7, wherein each subsequent prompt of the series of prompts is dynamically selected using the knowledge based diagnostic model based on a response to a preceding prompt of the series of prompts.

In a ninth aspect A9, the present disclosure provides the method according to any one of aspects A1-A8, wherein the knowledge based diagnostic model is an interactive model, and wherein assessing the risk of Addison's disease includes interacting with a user using the knowledge based diagnostic model to provide guidance on diagnosis and treatment of Addison's disease.

In a tenth aspect A10, the present disclosure provides the method according to any one of aspects A1-A9, wherein the method further comprises, in response to the assessed risk of Addison's disease being greater than a predetermined threshold, administering at least one of intravenous fluids, glucocorticoids, or mineralocorticoids.

In an eleventh aspect A11, the present disclosure provides a diagnostic system for assessing the risk of Addison's disease, the system comprising a memory configured to store instructions; and a processor communicatively connected to the memory and configured to execute the stored instructions to receive first medical training data; train a plurality of first machine learning models on the received first medical training data to generate an ensemble of diagnostic models; receive second medical training data; train a second machine learning model on the received second medical training data to generate a knowledge based diagnostic model; receive new patient data; determine whether the ensemble of diagnostic models indicates a risk for Addison's disease for the new patient data; and, in a case where the ensemble of diagnostic models indicates a risk for Addison's disease for the new patient data, assess the risk of Addison's disease using the knowledge based diagnostic model.

In a twelfth aspect A12, the present disclosure provides the system according to aspect A11, wherein the ensemble of diagnostic models includes one or more of a decision tree model, a random forest model, a neural network, a support vector machine, or a nearest neighbor classifier.

In a thirteenth aspect A13, the present disclosure provides the system according to any one of aspects A11-A12, wherein the plurality of first machine learning models is trained by selecting at least a first machine learning model and a second machine learning model for the ensemble of diagnostic models, wherein the second machine learning model is different from the first machine learning model; training the first machine learning model using a first subset of the received first medical training data to generate a first model of the ensemble of diagnostic models; and training the second machine learning model using a second subset of the received first medical training data to generate a second model of the ensemble of diagnostic models, wherein the second subset of the received first medical training data is different from the first subset of the received first medical training data, and wherein the risk for Addison's disease for the new patient data is determined based on outputs of the first machine learning model and the second machine learning model.

In a fourteenth aspect A14, the present disclosure provides the system according to aspect A13, wherein it is determined that the ensemble of diagnostic models indicates a risk for Addison's disease for the new patient data in a case where the outputs of either the first machine learning model or the second machine learning indicate the risk for Addison's disease for the new patient data.

In a fifteenth aspect A15, the present disclosure provides the system according to any one of aspects A11-A14, wherein the second machine learning model is trained by generating an expert model including a plurality of rules based on domain knowledge included in the received second medical training data; refining the plurality of rules in the expert model based on patient data included in the received second medical training data; and generating the knowledge based diagnostic model based on the refined plurality of rules.

In a sixteenth aspect A16, the present disclosure provides the system according to any one of aspects A11-A15, wherein the knowledge based diagnostic model includes one or more of a rules based model and a data driven machine learning model.

In a seventeenth aspect A17, the present disclosure provides the system according to any one of aspects A11-A16, wherein the risk of Addison's disease is assessed using the knowledge based diagnostic model by displaying, on a user interface, a series of prompts to receive further patient medical data; and assessing the risk of Addison's disease based the received further patient medical data in response to the series of prompts.

In an eighteenth aspect A18, the present disclosure provides the system according to aspect A17, wherein each subsequent prompt of the series of prompts is dynamically selected using the knowledge based diagnostic model based on a response to a preceding prompt of the series of prompts.

In a nineteenth aspect A19, the present disclosure provides the system according to any one of aspects A11-A18, wherein the knowledge based diagnostic model is an interactive model, and wherein the processor is configured to further execute the stored instructions to interact with a user using the knowledge based diagnostic model to provide guidance on diagnosis and treatment of Addison's disease.

In a twentieth aspect A20, the present disclosure provides non-transitory computer readable storage medium configured to store a program that executes a diagnostic method, the method comprising receiving first medical training data; training a plurality of first machine learning models on the received first medical training data to generate an ensemble of diagnostic models; receiving second medical training data; training a second machine learning model on the received second medical training data to generate a knowledge based diagnostic model; receiving new patient data; determining whether the ensemble of diagnostic models indicates a risk for Addison's disease for the new patient data; and, in a case where the ensemble of diagnostic models indicates a risk for Addison's disease for the new patient data, assessing the risk of Addison's disease using the knowledge based diagnostic model.

In a twenty-first aspect A21, the present disclosure provides a method, executed by a programmed data processing device system, of assessing the risk of Addison's disease, the method comprising receiving new patient data; receiving an ensemble of diagnostic models and a knowledge based diagnostic model; determining whether the ensemble of diagnostic models indicates a risk for Addison's disease for the new patient data; and, in a case where the ensemble of diagnostic models indicates a risk for Addison's disease for the new patient data, assessing the risk of Addison's disease using the knowledge based diagnostic model.

In a twenty-second aspect A22, the present disclosure provides the method according to aspect A21, wherein the ensemble of diagnostic models includes one or more of a decision tree model, a random forest model, a neural network, a support vector machine, or a nearest neighbor classifier.

In a twenty-third aspect A23, the present disclosure provides the method according to any one of aspects A21-A22, wherein the method further comprises receiving first training data; and training the ensemble of diagnostic models by selecting at least a first machine learning model and a second machine learning model, wherein the second machine learning model is different from the first machine learning model; training the first machine learning model using a first subset of the received first training data to generate a first diagnostic model; training the second machine learning model using a second subset of the received first training data to generate a second diagnostic model; and combining the first diagnostic model and the second diagnostic model into the ensemble of diagnostic models, wherein the risk for Addison's disease for the new patient data is determined based on an output of the ensemble of diagnostic models.

In a twenty-fourth aspect A24, the present disclosure provides the method according to aspect A23, wherein it is determined that the ensemble of diagnostic models indicates a risk for Addison's disease for the new patient data in a case where the outputs of either the first diagnostic model or the second diagnostic model indicate the risk for Addison's disease for the new patient data.

In a twenty-fifth aspect A25, the present disclosure provides the method according to any one of aspects A21-A24, wherein the method further comprises receiving second training data; and training the knowledge based diagnostic model by generating an expert model including a plurality of rules based on domain knowledge included in the received second medical training data; and refining the plurality of rules in the expert model based on patient data included in the received second medical training data.

In a twenty-sixth aspect A26, the present disclosure provides the method according to any one of aspects A21-A25, wherein the knowledge based diagnostic model includes one or more of a rule based model or a data driven machine learning model.

In a twenty-seventh aspect A27, the present disclosure provides the method according to any one of aspects A21-A26, wherein assessing the risk of Addison's disease using the knowledge based diagnostic model further includes displaying, on a user interface, a series of prompts to receive further patient medical data; and assessing the risk of Addison's disease based the received further patient medical data in response to the series of prompts.

In a twenty-eighth aspect A28, the present disclosure provides the method according to aspect A27, wherein each subsequent prompt of the series of prompts is dynamically selected using the knowledge based diagnostic model based on a response to a preceding prompt of the series of prompts.

In a twenty-ninth aspect A29, the present disclosure provides the method according to any one of aspects A21-A28, wherein the knowledge based diagnostic model is an interactive model, and wherein assessing the risk of Addison's disease includes interacting with a user using the knowledge based diagnostic model to provide guidance on diagnosis and treatment of Addison's disease.

In a thirtieth aspect A30, the present disclosure provides the method according to any one of aspects A21-A29, wherein the method further comprises, in response to the assessed risk of Addison's disease being greater than a predetermined threshold, administering at least one of intravenous fluids, glucocorticoids, or mineralocorticoids.

In a thirty-first aspect A31, the present disclosure provides a diagnostic system for assessing the risk of Addison's disease, the system comprising a memory configured to store instructions; and a processor communicatively connected to the memory and configured to execute the stored instructions to receive new patient data; receive an ensemble of diagnostic models and a knowledge based diagnostic model; determine whether the ensemble of diagnostic models indicates a risk for Addison's disease for the new patient data; and, in a case where the ensemble of diagnostic models indicates a risk for Addison's disease for the new patient data, assess the risk of Addison's disease using the knowledge based diagnostic model.

In a thirty-second aspect A32, the present disclosure provides a non-transitory computer readable storage medium configured to store a program that executes a diagnostic method, the method comprising receiving new patient data; receiving an ensemble of diagnostic models and a knowledge based diagnostic model; determining whether the ensemble of diagnostic models indicates a risk for Addison's disease for the new patient data; and, in a case where the ensemble of diagnostic models indicates a risk for Addison's disease for the new patient data, assessing the risk of Addison's disease using the knowledge based diagnostic model.

Subsets or combinations of various aspects of the disclosure described above provide further aspects of the disclosure.

These and other changes can be made to the invention in light of the above-detailed description and still fall within the scope of the present invention. In general, in the following claims, the terms used should not be construed to limit the invention to the specific aspects of the disclosure disclosed in the specification. Accordingly, the invention is not limited by the disclosure, but instead its scope is to be determined entirely by the following claims.

What is claimed is:

1. A processor executed method for assessing the risk of Addison's disease, comprising:
receiving first medical training data;
training a plurality of first machine learning models on the received first medical training data to generate an ensemble of diagnostic models;
receiving second medical training data;
training a second machine learning model on the received second medical training data to generate a knowledge based diagnostic model;
receiving new patient data;
determining whether the ensemble of diagnostic models indicates a risk for Addison's disease for the new patient data; and
in a case where the ensemble of diagnostic models indicates a risk for Addison's disease for the new patient data, assessing the risk of Addison's disease using the knowledge based diagnostic model.

2. The method according to claim 1, wherein the ensemble of diagnostic models includes one or more of a decision tree model, a random forest model, a neural network, a support vector machine, or a nearest neighbor classifier.

3. The method according to claim 1,
wherein training the plurality of first machine learning models includes:
selecting at least a first machine learning model and a second machine learning model for the ensemble of diagnostic models, wherein the second machine learning model is different from the first machine learning model;
training the first machine learning model using a first subset of the received first medical training data to generate a first model of the ensemble of diagnostic models; and
training the second machine learning model using a second subset of the received first medical training data to generate a second model of the ensemble of diagnostic models, wherein the second subset of the received first medical training data is different from the first subset of the received first medical training data, and
wherein the risk for Addison's disease for the new patient data is determined based on outputs of the first machine learning model and the second machine learning model.

4. The method according to claim 3, wherein, it is determined that the ensemble of diagnostic models indicates a risk for Addison's disease for the new patient data in a case where the outputs of either the first machine learning model or the second machine learning indicate the risk for Addison's disease for the new patient data.

5. The method according to claim 1,
wherein training the second machine learning model includes:
generating an expert model including a plurality of rules based on domain knowledge included in the received second medical training data;
refining the plurality of rules in the expert model based on patient data included in the received second medical training data; and
generating the knowledge based diagnostic model based on the refined plurality of rules.

6. The method according to claim 1, wherein the knowledge based diagnostic model includes one or more of a rules based model and a data driven machine learning model.

7. The method according to claim 1,
wherein assessing the risk of Addison's disease using the knowledge based diagnostic model further includes:
displaying, on a user interface, a series of prompts to receive further patient medical data; and
assessing the risk of Addison's disease based the received further patient medical data in response to the series of prompts.

8. The method according to claim 7, wherein each subsequent prompt of the series of prompts is dynamically selected using the knowledge based diagnostic model based on a response to a preceding prompt of the series of prompts.

9. The method according to claim 1,
wherein the knowledge based diagnostic model is an interactive model, and
wherein assessing the risk of Addison's disease includes interacting with a user using the knowledge based diagnostic model to provide guidance on diagnosis and treatment of Addison's disease.

10. The method according to claim 1, further comprising, in response to the assessed risk of Addison's disease being greater than a predetermined threshold, administering at least one of intravenous fluids, glucocorticoids, or mineralocorticoids.

11. A diagnostic system for assessing the risk of Addison's disease, the system comprising:
a memory configured to store instructions; and
a processor communicatively connected to the memory and configured to execute the stored instructions to:
receive first medical training data;
train a plurality of first machine learning models on the received first medical training data to generate an ensemble of diagnostic models;
receive second medical training data;
train a second machine learning model on the received second medical training data to generate a knowledge based diagnostic model;
receive new patient data;
determine whether the ensemble of diagnostic models indicates a risk for Addison's disease for the new patient data; and
in a case where the ensemble of diagnostic models indicates a risk for Addison's disease for the new patient data, assess the risk of Addison's disease using the knowledge based diagnostic model.

12. The system according to claim 11, wherein the ensemble of diagnostic models includes one or more of a decision tree model, a random forest model, a neural network, a support vector machine, or a nearest neighbor classifier.

13. The system according to claim 11,
the plurality of first machine learning models is trained by:
selecting at least a first machine learning model and a second machine learning model for the ensemble of diagnostic models, wherein the second machine learning model is different from the first machine learning model;
training the first machine learning model using a first subset of the received first medical training data to generate a first model of the ensemble of diagnostic models; and
training the second machine learning model using a second subset of the received first medical training data to generate a second model of the ensemble of diagnostic models, wherein the second subset of the received first medical training data is different from the first subset of the received first medical training data, and
wherein the risk for Addison's disease for the new patient data is determined based on outputs of the first machine learning model and the second machine learning model.

14. The system according to claim 13, wherein, it is determined that the ensemble of diagnostic models indicates a risk for Addison's disease for the new patient data in a case where the outputs of either the first machine learning model or the second machine learning indicate the risk for Addison's disease for the new patient data.

15. The system according to claim 11,
wherein the second machine learning model is trained by:
generating an expert model including a plurality of rules based on domain knowledge included in the received second medical training data;
refining the plurality of rules in the expert model based on patient data included in the received second medical training data; and
generating the knowledge based diagnostic model based on the refined plurality of rules.

16. The system according to claim 11, wherein the knowledge based diagnostic model includes one or more of a rules based model and a data driven machine learning model.

17. The system according to claim 11,
wherein the risk of Addison's disease is assessed using the knowledge based diagnostic model by:
displaying, on a user interface, a series of prompts to receive further patient medical data; and
assessing the risk of Addison's disease based the received further patient medical data in response to the series of prompts.

18. The system according to claim 17, wherein each subsequent prompt of the series of prompts is dynamically selected using the knowledge based diagnostic model based on a response to a preceding prompt of the series of prompts.

19. The system according to claim 11,
wherein the knowledge based diagnostic model is an interactive model, and
wherein the processor is configured to further execute the stored instructions to interact with a user using the knowledge based diagnostic model to provide guidance on diagnosis and treatment of Addison's disease.

20. A non-transitory computer readable storage medium configured to store a program that executes a diagnostic method, the method comprising:
receiving first medical training data;
training a plurality of first machine learning models on the received first medical training data to generate an ensemble of diagnostic models;
receiving second medical training data;
training a second machine learning model on the received second medical training data to generate a knowledge based diagnostic model;
receiving new patient data;
determining whether the ensemble of diagnostic models indicates a risk for Addison's disease for the new patient data; and
in a case where the ensemble of diagnostic models indicates a risk for Addison's disease for the new patient data, assessing the risk of Addison's disease using the knowledge based diagnostic model.
